(19) United States
(12) Patent Application Publication
(10) Pub. No.: US 2016/0007879 A1
(43) Pub. Date: Jan. 14, 2016

(54) MULTIFREQUENCY SIGNAL PROCESSING CLASSIFIERS FOR DETERMINING A TISSUE CONDITION

(71) Applicant: THE REGENTS OF THE UNIVERSITY OF CALIFORNIA, Oakland, CA (US)

(72) Inventors: Cesar A. GONZALEZ, Anahuac (MX); Boris RUBINSKY, El Cerrito, CA (US)

(73) Assignee: THE REGENTS OF THE UNIVERSITY OF CALIFORNIA, Oakland, CA (US)

(21) Appl. No.: 14/772,299

(22) PCT Filed: Mar. 14, 2014
(86) PCT No.: PCT/US14/27268

§ 371 Date: Sep. 2, 2015

Related U.S. Application Data
(60) Provisional application No. 61/788,858, filed on Mar. 15, 2013.
(60) Provisional application No. 61/810,846, filed on Apr. 11, 2013.

(51) Int. Cl. A61B 5/053; A61B 5/00; A61B 5/02

(52) U.S. Cl. CPC A61B 5/0537; A61B 5/02042; A61B 5/4878; A61B 5/443; A61B 5/4519; A61B 5/4848; A61B 5/4064

(57) ABSTRACT

Volumetric Electromagnetic Phase Shift Spectroscopy (VEPS)-based methods of analyzing a tissue are provided. Aspects of the methods comprise obtaining a VEPS-based tissue classifier, or “signature” for a tissue at a single point in time. These methods find particular use in non-invasively determining the condition of a tissue, e.g. brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, trunk tissue, prostate tissue, breast tissue, esophagus tissue, GI tract tissue, etc., in an individual. Devices and systems thereof that find use in practicing the subject methods are also provided.

### FIELD OF THE INVENTION

This invention pertains to the use of bioelectrical impedance to determine the condition of a tissue.

### BACKGROUND OF THE INVENTION

A number of different medical conditions—edema, hemorrhage, hematoma, ischemia, dehydration, the presence of a tumor, infection, brain degeneration, extravasation, internal bleeding, maternal hemorrhage, and the like—are associated with abnormal tissue water content and water content distribution. Biological tissues contain compounds with measurable electrical properties such as the intracellular and extracellular ionic solutions, the capacitative cell membrane, charged macromolecules and polar water. The combination of these compounds in terms of composition and structure affect the overall electromagnetic properties of the tissue. As such, technologies have been developed that detect abnormal tissue water content and, hence, these medical conditions, by assessing the overall electromagnetic properties of the tissue.

One technology of particular interest is Volumetric Electromagnetic Phase Shift Spectroscopy (VEPS). In applications of VEPS to analyzing a tissue, bioimpedence analysis based on the conduction of an applied electrical current in the tissue is employed to detect a variety of medical conditions. Specifically, a change is detected in the phase angle between the AC currents in an emitting and a sensing induction coil or antenna over a wide range (i.e. a spectrum) of frequencies when a volume of tissue is placed between the emitting and a sensing coil or antenna through which AC currents are passed. This spectroscopic measuring method is simpler and more reliable than other methods for detecting bioimpedence properties of a tissue. For example, the method does not require galvanic coupling between the electrode and the skin or the tissue under measurement. Instead, the VEPS system is completely non-invasive. In addition, instantaneous measurements of the phase shift may be made. Alternatively, measurements may be made over time, e.g. to detect the progress of the phase shift in time to determine the development of the medical condition. VEPS and how to record VEPS measurements is described in greater detail in U.S. Pat. No. 7,638,341, U.S. Pat. No. 7,910,374, U.S. Pat. No. 8,101,421, and U.S. Pat. No. 8,361,391, the full disclosures of which are incorporated herein by reference.

Typically, VEPS is performed to obtain a spectrum of phase shifts over a range of frequencies, which can be compared to spectrums obtained from the same tissue over several time points to determine if a medical condition is developing. What is needed, however, is a classification system for classifying a tissue condition based upon electromagnetic properties of the tissue obtained at a single recording session. The present invention addresses these issues.

### SUMMARY OF THE INVENTION

Volumetric Electromagnetic Phase Shift Spectroscopy (VEPS)-based methods of analyzing a tissue are provided. Aspects of the methods comprise obtaining a VEPS-based tissue classifier, or “signature” for a tissue at a single point in time. These methods find particular use in non-invasively determining the condition of a tissue, e.g. brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, trunk tissue, prostate tissue, breast tissue, esophagus tissue, GI tract tissue, etc., in an individual. Devices and systems thereof that find use in practicing the subject methods are also provided.

In some aspects of the invention, a method of obtaining a VEPS tissue signature is provided. In some embodiments, the method comprises positioning a tissue between a first induction coil and a second induction coil; driving an alternating current in a frequency range through the first induction coil; measuring the alternating current produced in the second induction coil at the frequency range, and determining a phase shift of the alternating current between the first induction coil and the second induction coil at the frequency range to obtain a VEPS tissue signature. In some embodiments, the method further comprises driving an alternating current in a second frequency range through the first induction coil; measuring the alternating current produced in the second induction coil at the second frequency range; determining a phase shift of the alternating current between the first induction coil and the second induction coil at the second frequency range; and obtaining a VEPS tissue signature based on the first frequency range and the second frequency range. In some embodiments, the first and/or second frequency range is within between 1 Hz and 1 THz. In some embodiments, the first and/or second frequency range is in the range of between 1 KHz to 20 GHz. In some embodiments, the first and/or second frequency range is within between 0.1 MHz and 150 MHz. In some embodiments, the first and/or second frequency range is in the range of between 1 KHz to 20 GHz. In some embodiments the first and/or second frequency range is in the range of between 100 MHz and 500 MHz.

In some embodiments, the first and second induction coils do not contact the tissue. In some embodiments, the tissue is selected from the group consisting of: brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, prostate tissue, breast tissue, esophagus tissue, GI tract tissue and trunk tissue.

In some aspects of the invention, a method is provided for providing a determination of the condition of a tissue in a subject. In some embodiments, the method comprises obtaining a VEPS tissue signature, and determining the condition of a tissue in a subject based on the tissue signature. In some embodiments, the condition is selected from the group consisting of: edema, hemorrhage, hematoma, ischemia, dehydration, the presence of a tumor, infection, brain degeneration, extravasation, internal bleeding, maternal hemorrhage, and tissue health relative to age. In some embodiments, the determining step comprises comparing the VEPS tissue signature to a reference, and providing a determination based on the comparison. In certain embodiments, the comparing comprises graphically plotting the tissue signature relative to a panel of classifiers. In some embodiments, the method further comprises determining a clinical parameter. In some embodiments, the clinical parameter is the age of the subject.

In some embodiments, the determination is used to provide a diagnosis for the subject, wherein the method further comprises providing a diagnosis for the subject based on the determination of the condition of the tissue. In some embodiments, the determination is used to provide a prognosis for the subject, wherein the method further comprises providing a prognosis for the subject based on the determination of the condition of the tissue. In some embodiments, the determination is used to monitor a subject's health or responsiveness to a therapeutic treatment, wherein the method further comprises obtaining a second VEPS signature at a second point in time, and monitoring the subject's health or responsiveness to a therapeutic treatment based on the determination of the first VEPS signature and the second VEPS signature.

In some aspects of the invention, a system for obtaining a VEPS tissue signature is provided. In some embodiments, the system comprises a first induction coil and a second induction coil positioned opposite one another; and a measurement system operably connected to the second induction coil, wherein the measurement system is configured to measure a phase shift of one or more alternating currents between the first and second induction coil at one or more frequencies in two or more frequency ranges. In some embodiments, at least one of the two or more frequency ranges is in the range of between 1 Hz and 1 THz. In some embodiments, at least one of the two or more frequency ranges is in the range of between 1 KHz to 20 GHz. In some embodiments, at least one of the two or more frequency ranges is in the range of between 0.1 MHz and 150 MHz. In some embodiments, at least one of the two or more frequency ranges is in the range of between 1 KHz to 20 GHz. In some embodiments, at least one of the two or more frequency ranges is in the range of between 100 MHz and 500 MHz.

In some embodiments, the first and second induction coils do not contact the tissue. In some embodiments, the tissue is selected from the group consisting of: brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, prostate tissue, breast tissue, esophagus tissue, GI tract tissue and trunk tissue. In some embodiments, the system further comprises a data processor module configured to calculate VEPS values from the plurality of frequencies corresponding to the two or more frequency ranges.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Schematic of the VEPS Head/coil configuration and a block diagram of the experimental prototype. The system consists of five modules: digital synthesizer, transceiver, phase detector, data acquisition and data processing.

FIG. 2. Photographs of the VEPS clinical Head/coil device and an illustration of a patient in a critical care unit wearing the device.

FIG. 3. Flow diagram of the clinical study.

FIG. 4. Computer tomography (CT) of the brain of the patients involved in the study, prior to the VEPS measurements. The CT's are divided into two groups according to clinical neurology pathology valuation: Edema and Hematoma. Moderate to severe diffuse brain edema without hemorrhage or hematoma, and subdural or epidural wall haematoma regions are evident. A description of the particular pathology is given next to each CT image.

FIG. 5. The β value for all the subjects of this study as a function of the subject age. Healthy volunteers, patients with brain condition of edema and of hematoma are marked with different symbols.

FIG. 6. The γ value for all the subjects of this study as a function of age. Healthy volunteers, patients with brain condition of edema and of hematoma are marked with different symbols.

FIG. 7. A scalar classifier plot of each experimental subject in terms of two values for that subject, β and γ. Each data point represents a subject. Healthy volunteers, patients with brain condition of edema and of hematoma are marked with different symbols.

### DETAILED DESCRIPTION OF THE INVENTION

Volumetric Electromagnetic Phase Shift Spectroscopy (VEPS)-based methods of analyzing a tissue are provided. Aspects of the methods comprise obtaining a VEPS-based tissue classifier, or “signature” for a tissue. These methods find particular use in non-invasively determining the condition of a tissue, e.g. brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, trunk tissue, prostate tissue, breast tissue, esophagus tissue, GI tract tissue, etc., in an individual. Devices and systems thereof that find use in practicing the subject methods are also provided. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the cell” includes reference to one or more cells and equivalents thereof, as known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Methods

In some aspects of the invention, methods, devices and systems thereof are provided for determining the condition of a tissue. Embodiments of the subject invention are directed to measuring electromagnetic properties of the tissue. Of particularly interest in these embodiments is measuring the bioelectrical impedance, or “bioimpedence”, of the tissue to an externally applied electric current, e.g. phase shifts, shifts in amplitude, shifts in wavelength, and the like. In further describing aspects of the invention, the following description focuses on determining the condition of a tissue by measuring phase shifts using Volumetric Electromagnetic Phase Shift Spectroscopy (VEPS). However, the ordinarily skilled artisan will readily appreciate that the subject methods, devices and systems also encompass determining the condition of a tissue by measuring changes in other wave characteristics as a result of bioelectrical impedance, e.g. as described herein or known in the art.

In some aspects of the invention, methods are provided for determining the condition of a tissue that rely on the use of Volumetric Electromagnetic Phase Shift Spectroscopy (VEPS). By “Volumetric Electromagnetic Phase Shift Spectroscopy”, or “VEPS”, it is meant the electrical measurement system that detects a phase shift between applied and measured currents across a bulk tissue. VEPS can detect tissue properties inside the body through non-contact electromagnetic measurements from the exterior of the body, thereby providing bulk information on the properties of an organ or tissue. VEPS and the general application of VEPS to detect tissue properties are well known in the art. See, for example, U.S. Pat. No. 7,638,341, U.S. Pat. No. 7,910,374, U.S. Pat. No. 8,101,421, and U.S. Pat. No. 8,361,391, the full disclosures of which are incorporated herein by reference. In practicing the subject methods, VEPS-based measurements of one or a range of frequencies are employed to obtain VEPS-based classifiers, or “signatures” for a tissue. By a “VEPS-based tissue classifier”, or “VEPS-based tissue signature”, it is meant a VEPS single value or combination of values that is characteristic of, i.e. a “signature” for, a tissue condition and may be used to classify the tissue under study as having that condition, e.g. a VEPS value representative of reading(s) within an α, β, γ, etc. and other ranges of relevant frequencies; in some instances, a combination of two or more VEPS values representative of reading(s) within each of two ranges of relevant frequencies, e.g. (, β), (β, γ), (, γ), etc., in certain instances three or more VEPS values representative of reading(s) within each of three relevant ranges of frequencies, i.e. (α, β, γ) and so on.

The disclosed methods for determining the condition of a tissue are based in part on the discovery by the inventors of a new technique for analyzing the multifrequency data of VEPS. The inventors have discovered the certain frequencies, when analyzed alone or in combination, can produce an immediate characterization of a tissue, and hence, of a medical condition. In other words, VEPS data from one relevant frequency (or one range of frequencies), or two or more relevant frequencies (or two or more relevant ranges of frequencies) in combination may be used as a tissue classifier, or signature, to immediately identify a pathological condition in organ or tissue. As such, aspects of the invention provide methods for analyzing a tissue at a single time-point to obtain a VEPS tissue signature, and using a VEPS tissue signature to determine the condition of a tissue, which may in turn be used to diagnose a medical condition, provide a prognosis of a medical condition, predict the responsiveness of a tissue to a medical treatment, and the like.

In practicing the subject methods, a VEPS-based tissue signature is obtained by detecting a phase shift in at least one relevant range of frequencies to arrive at least one VEPS value, and using the at least one VEPS value so obtained to obtain the VEPS signature. In some instances, the phase shift is detected at a plurality of ranges of frequencies, i.e. frequencies in 2 ranges, frequencies in 3 ranges, frequencies in 4 ranges, etc.—to arrive at a plurality of corresponding VEPS values, and the plurality of VEPS values so obtained are used in combination to obtain the VEPS signature.

For example, a tissue is positioned between a first induction coil and a second induction coil; alternating current within a first frequency range is driven through the first induction coil; and the alternating current produced in the second induction coil is measured. The phase shift of the alternating current between the first induction coil and the second induction coil at the first frequency range may then be determined to arrive at a first VEPS value, wherein the phase shift is caused by the presence of the tissue located between the first and second induction coils. This VEPS value may be used as a VEPS-based signature. In some embodiments, a phase shift at a second frequency range may also be determined, e.g. alternating current within a second frequency range is driven through the first induction coil; the alternating current produced in the second induction coil is measured; and the phase shift of the alternating current between the first induction coil and the second induction coil at the second frequency range determined to arrive at a second VEPS value; where the two VEPS values in combination (i.e. paired together) make up the VEPS signature. In some embodiments, a phase shift at a third frequency range may also be determined, i.e. alternating current within a third frequency range is driven through the first induction coil; the alternating current produced in the second induction coil is measured; and the phase shift of the alternating current between the first induction coil and the second induction coil at the third frequency range determined to arrive at a third VEPS value; where the three VEPS values, in combination make up the VEPS signature.

In some embodiments, antennae are used in place of induction coils. Thus, for example, a tissue may be placed between a first antenna and a second antenna, voltage within a first frequency range is driven through the first antenna, and the voltage that is produced in the second antenna is measured. The phase shift of the voltage between the first antenna and the second antenna at the first frequency range may then be determined to arrive at a first VEPS value, this VEPS value making up the VEPS-based signature. In some embodiments, a phase shift at a second frequency range may also be determined, e.g. voltage within a second frequency range is driven through the first antenna; the voltage produced in the second antenna is measured; and the phase shift of the voltage between the first antenna and the second antenna at the second frequency range determined to arrive at a second VEPS value; where the two VEPS values in combination (i.e. paired together) make up the VEPS signature. In some embodiments, a phase shift at a third frequency range may also be determined, i.e. voltage within a third frequency range is driven through the first antenna; the voltage produced in the second antenna is measured; and the phase shift of the voltage between the first antenna and the second antenna at the third frequency range determined to arrive at a third VEPS value; where the three VEPS values, in combination make up the VEPS signature.

As indicated above, in some instances, alternating currents (or voltage) at a plurality of frequencies within a designated frequency range is driven through the first induction coil (or antenna), and the alternating current (or voltages) that is produced in the second induction coil (or antenna) at the plurality of frequencies with the designated range are measured. In such instances, the phase shifts at the plurality of frequencies are calculated and integrated, e.g. by summing the values, to obtain a single VEPS value, i.e. the VEPS value representative of that frequency range. For example, for a frequency range of 20 MHz to 40 MHz, the phase shift may be determined for a plurality of frequencies selected from, e.g., 20 MHz, 21 MHz, 22 MHz, 23 MHz, 24 MHz, 25 MHz, 26 MHz, 27 MHz, 28 MHz, 29 MHz, 20 MHz, 31 MHz, 32 MHz, 33 MHz, 34 MHz, 35 MHz, 36 MHz, 37 MHz, 38 MHz, 39 MHz, and 40 MHz, and the measurements integrated to arrive at a single VEPS value representative of the 20 MHz to 40 MHz range. As another example, for a frequency range of 150 MHz to 170 MHz, the phase shift may be determined for a plurality of frequencies selected from, e.g., 150 MHz, 151 MHz, 152 MHz, 153 MHz, 154 MHz, 155 MHz, 156 MHz, 157 MHz, 158 MHz, 159 MHz, 160 MHz, 161 MHz, 162 MHz, 163 MHz, 164 MHz, 165 MHz, 166 MHz, 167 MHz, 168 MHz, 169 MHz, and 170 MHz, and the measurements integrated to arrive at a single VEPS value representative of the 150 MHz to 170 MHz range. In some instances, phase shifts at 2 or more frequencies in the range are detected and integrated into a single VEPS value; in some instances, phase shifts at 3, 4, or 5 or more frequencies are measured and integrated; in some instances, phase shifts at 6, 7, 8, 9, or 10 frequencies or more are measured and integrated, e.g. 11, 12, 13, 14, 15, 16, 17, 18, 19, or 20 or more are measured and integrated into a single VEPS value. In some instances, phase shifts for all of the frequencies in the range are measured, and the phase shifts are integrated into a single VEPS value representative of that range.

In other instances, alternating current (or voltage) at a single frequency within a designated frequency range is driven through the first induction coil (or antenna), the alternating current (or voltage) that is produced in the second induction coil (or antenna) at this frequency is measured, the phase shift for this frequency is calculated, and the calculated phase shift used as the VEPS value, i.e. the VEPS value representative of that frequency range. For example, for a frequency range of 20 MHz to 40 MHz, the phase shift may be determined for a single frequency selected from, e.g., 20 MHz, 21 MHz, 22 MHz, 23 MHz, 24 MHz, 25 MHz, 26 MHz, 27 MHz, 28 MHz, 29 MHz, 30 MHz, 31 MHz, 32 MHz, 33 MHz, 34 MHz, 35 MHz, 36 MHz, 37 MHz, 38 MHz, 39 MHz, and 40 MHz, and the phase shift used as the VEPS value representative of the 20 MHz to 40 MHz range. As another example, for a frequency range of 150 MHz to 170 MHz, the phase shift may be determined for a single frequency selected from, e.g., 150 MHz, 151 MHz, 152 MHz, 153 MHz, 154 MHz, 155 MHz, 156 MHz, 157 MHz, 158 MHz, 159 MHz, 160 MHz, 161 MHz, 162 MHz, 163 MHz, 164 MHz, 165 MHz, 166 MHz, 167 MHz, 168 MHz, 169 MHz, and 170 MHz and the phase shift used as the VEPS value representative of the 150 MHz to 170 MHz range.

The phase shifts for any frequency or range of frequencies may be employed in determining a VEPS value to obtain a VEPS signature. In some instances, the frequencies are in the range of between 1 Hz and 1 THz. In some such instances, the frequencies are in the range of between 1 KHz to 20 GHz. In some such instances, the frequencies are in the range of between 10 KHz to 10 GHz. In certain instances, the frequencies are in the range of between 1 MHz and 10 GHz. Frequencies of particular interest for sampling to obtain a VEPS value are frequencies of alternating current in the range of between 0.1 MHz and 150 MHz, of between 0.5 and 100 MHz, of between 1 MHz and 70 MHz, of between 10 MHz and 60 MHz, of between 20 MHz and 50 MHz, of between 25 MHz and 40 MHz, of between 30 MHz and 35 MHz, i.e. about 33 MHz. Also of particular interest in sampling are frequencies of alternating current in the range of between 100 MHz and 500 MHz, e.g. of between 120 MHz and 200 MHz, of between 130 MHz and 190 MHz; of between 140 MHz and 180 MHz; of between 130 MHz and 190 MHz; of between 140 MHz and 180 MHz; of between 150 MHz and 170 MHz; e.g. of between 155 MHz and 165 MHz, i.e. about 160 MHz. Typically, the first frequency range and the second frequency range do not overlap.

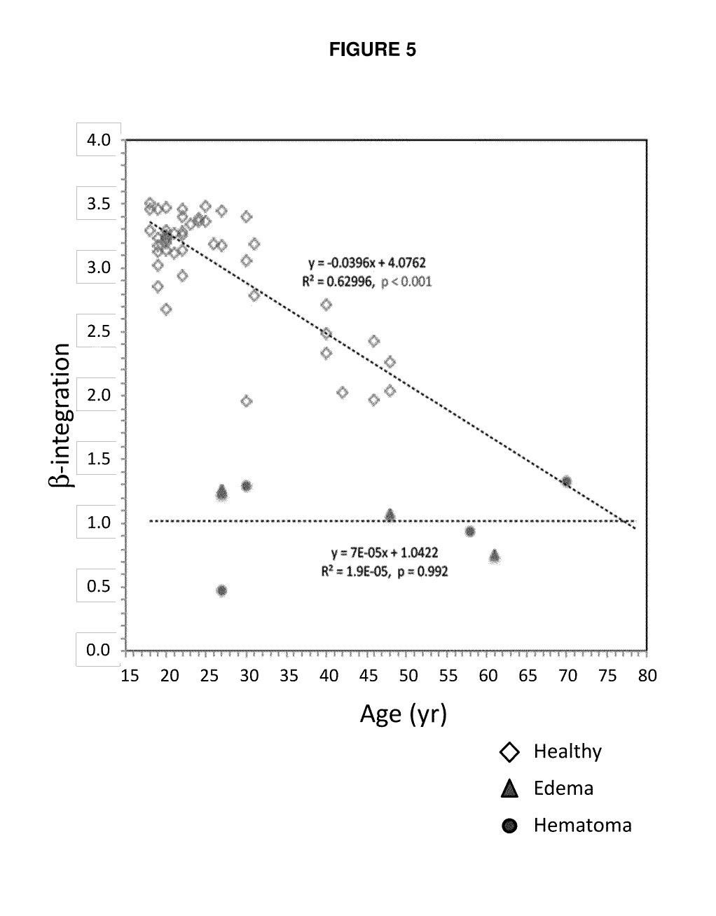

For example, as demonstrated by the working examples disclosed herein (see, e.g., FIGS. 5-7), a VEPS signature comprising a VEPS value in a β frequency range of about 20 MHz-40 MHz, e.g. about 33 MHz, and/or a VEPS value in a γ frequency range of about 150 MHz-170 MHz, e.g. about 160 MHz, may be used to identify a pathological condition such as edema, hematoma, or prematurely aging tissue, even before the patient is brought to a medical imaging facility. Thus, by summing for a subject the phase shift in brain tissue at frequencies in a β frequency range of from about 26 MHz to about 39 MHz to arrive at a β VEPS value for that subject, and/or summing for a subject the phase shift at frequencies in a γ frequency range of from about 153 MHz to about 166 MHz to arrive at a γ VEPS value for that subject, a VEPS signature may be arrived at that finds use in determining the health of the brain tissue.

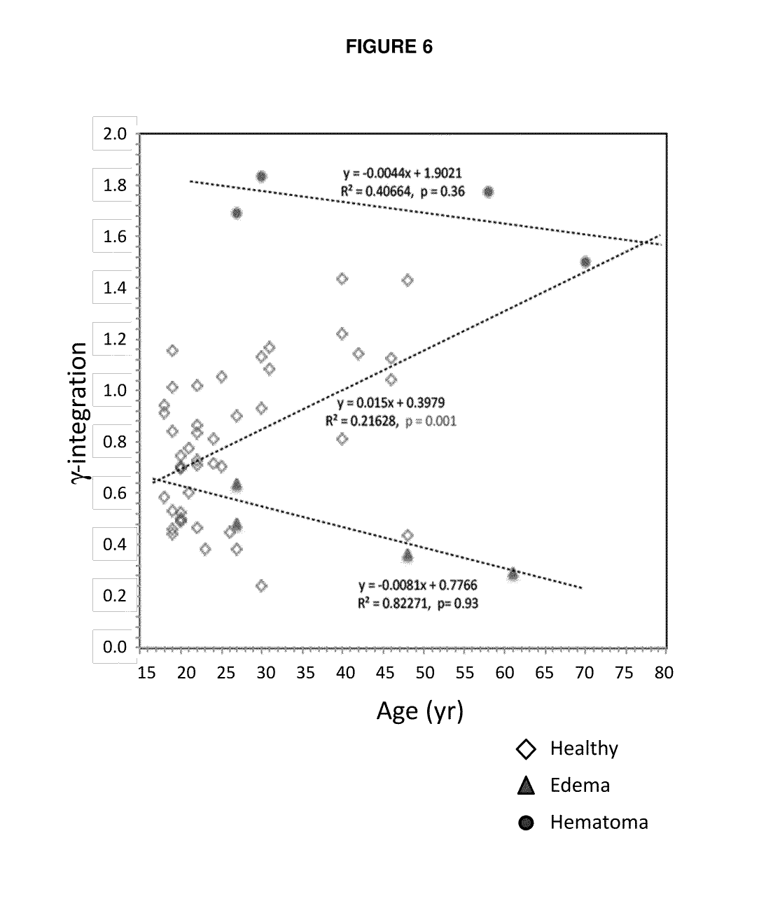

For example, FIG. 5, which depicts β value VEPS signatures as a function of age and medical condition, demonstrates that by analyzing β values alone in the context of an individual's age it is possible to identify a healthy brain versus a diseased brain at most ages (the exception being a healthy brain over age 75, where extrapolations from the data suggest that a healthy brain β value at the age of about 77 is comparable to that of a diseased brain at any age). Thus, a single value VEPS signature may be used to determine if a brain tissue is healthy (e.g. in a 15-35 year old, a β value of about 2.5 or more arrived at by summing the β values in the β frequency range of 26 MHz to 39 MHz; in a 35-60 year old, a β value of about 1.5 or more arrived at by summing the β values in the β frequency range of 26 MHz to 39 MHz) or may be aging prematurely (e.g. in a 15-35 year old arrived at by summing the β values in the β frequency range of 26 MHz to 39 MHz, a β value of less than about 2.5; in a 35-55 year old arrived at by summing the β values in the β frequency range of 26 MHz to 39 MHz, a β value of less than about 1.5 arrived at by summing the β values in the β frequency range of 26 MHz to 39 MHz). FIG. 6 demonstrates a similar utility for γ values measured alone. It is seen that the γ reading in normal brains changes with age, but with a different slope than that for the 13 readings. Thus, a VEPS value in a single frequency or a single narrow range of frequencies may be used as a VEPS signature of a healthy tissue versus diseased tissue for many subjects.

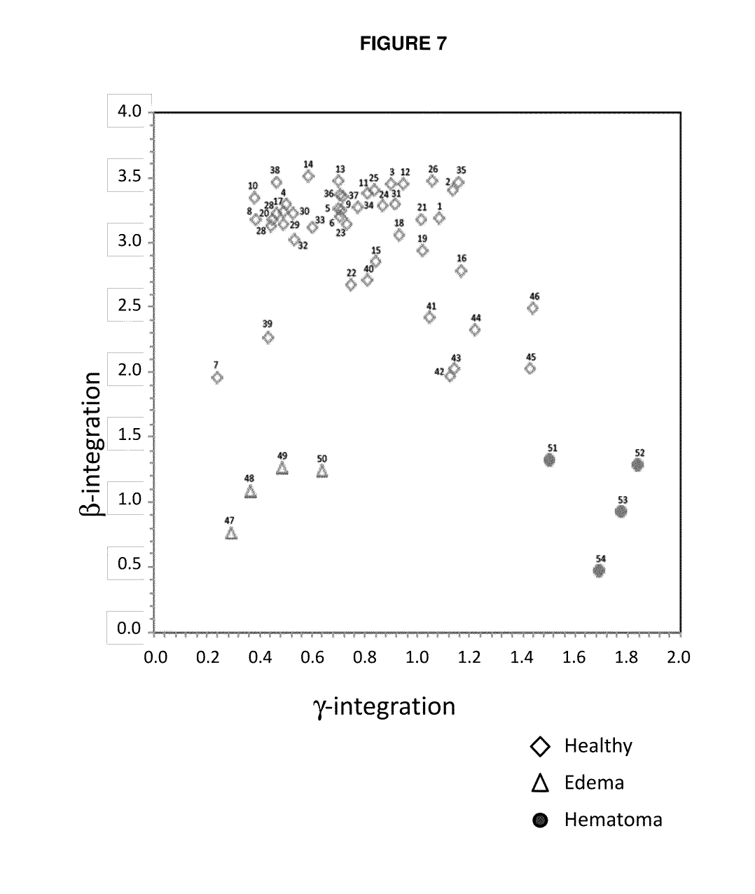

As another example, FIG. 7, which depicts VEPS signatures comprising paired β and γ values plotted on a β and γ graph, demonstrates the use of a two-value VEPS signature to identify a healthy brain versus a generally diseased brain, and moreover, the type of disease. FIG. 7 illustrates that a 13 value of about 1.5 or more and any γ value indicates a healthy brain, whereas a 13 value of less than about 1.5 and any γ value indicates a diseased brain. FIG. 7 also illustrates that further consideration of the γ value may be used to enhance the diagnosis, wherein a β value of less than about 1.5 and a γ value of less than about 1.2 indicates that the disease is edema, while a β value of less than about 1.5 and a γ value of about 1.2 or more indicates that the disease is hematoma. This analysis in FIG. 7 is an example of computer learning algorithms known as classifier analysis; see e.g. (B. Scholkopf and A. J. Smola, Learning with Kernels: Support Vector Machines, Regularization, Optimization, and Beyond. Cambridge, Mass.: MIT Press, 2002).

Other relevant frequencies and frequency ranges, e.g., from 1 Hz to 1 THz, from 1 Hz to 20 GHz, from 10 KHz to 10 GHz, and the like, may be readily determined by the ordinarily skilled artisan, e.g. as known in the art or as described herein. For example, a non-parametric statistical Mann-Whitney U test may be used to compare data from healthy volunteers with the data from patients with different medical conditions to identify which frequency or frequency range is relevant for a certain use and medical condition.

In some instances, the change in amplitude, or “amplitude shift”, of the electric current between the two coils or antennae may be measured, e.g. instead of or in addition to the phase shift, to arrive at the tissue signature. In other words, determining the condition of a tissue may comprise obtaining an “amplitude signature”. For example, a tissue is positioned between a first induction coil and a second induction coil; alternating current within a first frequency range is driven through the first induction coil; and the amplitude of the alternating current produced in the second induction coil is measured. The amplitude shift of the alternating current between the first induction coil and the second induction coil at the first frequency range may then be determined to arrive at a first amplitude value, wherein the amplitude shift is caused by the presence of the tissue located between the first and second induction coils. This value may be used as an amplitude signature. In some embodiments, an amplitude shift at a second frequency range may also be determined, e.g. alternating current within a second frequency range is driven through the first induction coil; the alternating current produced in the second induction coil is measured; and the amplitude shift of the alternating current between the first induction coil and the second induction coil at the second frequency range determined to arrive at a second amplitude value; where the two amplitude values in combination (i.e. paired together) make up the amplitude signature. In some embodiments, an amplitude shift at a third frequency range may also be determined, i.e. alternating current within a third frequency range is driven through the first induction coil; the alternating current produced in the second induction coil is measured; and the amplitude shift of the alternating current between the first induction coil and the second induction coil at the third frequency range determined to arrive at a third amplitude value; where the three amplitude values, in combination make up the amplitude signature. As such, in some embodiments, the method comprises determining an amplitude shift of the alternating current between the first induction coil and the second induction coil at the frequency range to obtain an amplitude signature, e.g. as described above.

In some instances, the method comprises using both a VEPS signature and an amplitude signature to determine the condition of the tissue. In other words, the method comprises determining a phase shift of the alternating current between the first induction coil and the second induction coil at the frequency range to obtain a VEPS signature; determining an amplitude shift of the alternating current between the first induction coil and the second induction coil at the frequency range to obtain an amplitude signature; and determining the condition of the tissue based on the VEPS signature and the amplitude signature.

In some embodiments, the method comprises using an amplitude shift to determine a phase shift, and using that phase shift to determine the condition of the tissue. In other words, the method comprises determining an amplitude shift of the alternating current between the first induction coil and the second induction coil at the frequency range, converting the amplitude shift into a phase shift, obtaining a VEPS signature based the phase shift; and determining the condition of the tissue based on the VEPS signature. Any convenient method or algorithm for calculating phase shift from amplitude may be employed.

In some instances, the subject methods of analyzing a tissue and obtaining a VEPS tissue signature for a subject further comprise providing the VEPS tissue signature as a report. In other words, the subject methods comprises determining the VEPS value at a first frequency (or range of frequencies), determining the VEPS value at a second frequency (or range of frequencies), and providing, i.e. generating, a report that includes the VEPS tissue signature. Thus, a subject method may further include a step of generating or outputting a report providing the results of a VEPS evaluation the sample, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

A VEPS tissue signature that is so obtained may then be employed in the clinic, e.g. in methods for determining a tissue condition and for diagnosing, prognosing, or treating a medical condition. For example, the VEPS tissue signature may be employed to diagnose edema, hemorrhage, hematoma, ischemia, dehydration, the presence of a tumor, infection, tissue degeneration (e.g. neurodegeneration), extravasation, internal bleeding, maternal hemorrhage, and the like; to characterize a diagnosed edema, hemorrhage, hematoma, ischemia, dehydration, the presence of a tumor, infection, brain degeneration, extravasation, internal bleeding, maternal hemorrhage, and the like; to determine a therapy for edema, hemorrhage, hematoma, ischemia, dehydration, the presence of a tumor, infection, brain degeneration, extravasation, internal bleeding, maternal hemorrhage, and the like; to monitor the responsiveness of an affected tissue to treatment for edema, hemorrhage, hematoma, ischemia, dehydration, the presence of a tumor, infection, brain degeneration, extravasation, internal bleeding, maternal hemorrhage, and the like, etc. as described herein. In other words, a medical practitioner will be able to provide a diagnosis, prognosis, or treatment for a tissue condition or monitor a tissue condition based upon the obtained VEPS tissue signature.

In some embodiments, the VEPS tissue signature is employed by comparing it to a reference, to identify similarities or differences with the reference, where the similarities or differences that are identified are then employed to diagnose a tissue condition in an individual, to characterize a diagnosed tissue condition, to monitor the responsiveness of the tissue condition to treatment for the condition, etc. For example, a reference may be a VEPS tissue signature that is representative of a tissue condition (i.e. a positive control) or that is representative of a healthy condition (i.e. a negative reference), which may be used, for example, as a reference/control in the evaluation of the VEPS signature for a given patient. As indicated above, the reference may be a positive reference/control, e.g., a VEPS tissue signature that is characteristic of a tissue condition, e.g. edema, hemorrhage, hematoma, ischemia, dehydration, the presence of a tumor, infection, brain degeneration, extravasation, internal bleeding, maternal hemorrhage, and the like. Alternatively, the reference may be a negative reference/control, e.g. a VEPS signature from a healthy tissue. References are preferably obtained from the same type of sample as the sample being analyzed. For example, if the brain of an individual is being evaluated, the reference/control would preferably be a VEPS tissue classifier from a brain.

In certain embodiments, the obtained VEPS tissue signature is compared to two or more references. For example, the obtained VEPS tissue signature may be compared to a negative reference and a positive reference to obtain confirmed information regarding the tissue condition. As another example, the obtained VEPS tissue signature may be compared to a reference that is representative of one condition, e.g. edema, and a reference that is representative of a second condition, e.g. hematoma.

The comparison of the obtained VEPS tissue signature and the one or more references may be performed using any convenient methodology, where a variety of methodologies are known to those of skill in the art. For example, those of skill in the art of classifiers will know that classifiers may be compared graphically, e.g. as a dot plot, in which the values for the first parameter of the classifier (e.g. VEPS values for the first frequency range) are plotted along the first axis, values for the second parameter of the classifier (e.g. VEPS values for the second frequency range) are plotted along the second axis, and specific regions/quadrants of the plot are identified by reference to a panel of VESP tissue signatures as being associated with specific tissue conditions. See, for example, FIG. 5, wherein VEPS tissue signatures comprising a low VEPS value for the beta frequency and a low VEPS value for the gamma frequency are indicative of edema, while VEPS signatures comprising a low VEPS value for the beta frequency and a high VEPS value for the gamma frequency are indicative of haematoma.

Depending on the type and nature of the reference/control profile(s) to which the obtained VEPS tissue signature is compared, the above comparison step yields a variety of different types of information regarding the tissue that is assayed. As such, such a comparison step can yield a positive/negative diagnosis of a tissue condition. Alternatively, such a comparison step can provide a characterization of a tissue condition, a prognosis of a tissue condition, or monitor a tissue condition.

In some embodiments, other analyses may be employed in conjunction with the aforementioned VEPS tissue signature to provide a tissue diagnosis for the individual. Such analyses are well known in the art, and include, for example, detecting one or more clinical parameters, e.g. age, weight, risk factors associated with the disease or disorder, and the like, and providing a diagnosis/prognosis/prediction of responsiveness to e.g. therapy based on the VEPS and these one or more clinical parameters.

In some embodiments, the subject methods of characterizing a tissue, diagnosing a medical condition, and the like include providing a characterization of the tissue, diagnosis of a medical condition, etc. In some such embodiments, the characterization or diagnosis may be provided by providing, i.e. generating, a written report that includes the practitioner's monitoring assessment, e.g. the practitioner's characterization of the subject's tissue (a “tissue characterization”), the practitioner's diagnosis of the subject's medical condition (a “diagnosis of a medical condition”), etc. Thus, a subject method may further include a step of generating or outputting a report providing the results of a monitoring assessment, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

### Reports

A “report,” as described herein, is an electronic or tangible document which includes report elements that provide information of interest relating to a subject monitoring assessment and its results. In some embodiments, a subject report includes at least a VEPS signature, e.g. as an aspect of the subject methods directed to obtaining a VEPS tissue signature, discussed in greater detail above. In some embodiments, a subject report includes at least a characterization of a tissue condition, i.e. a classification as edemous, as having a hematoma, as hemorrhagic, as ischemic, as comprising a tumor, etc., a diagnosis of a medical condition e.g. as an aspect of the subject methods directed to characterizing a tissue or providing a medical diagnosis for an individual, discussed in greater detail above. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a VEPS tissue signature for the tissue analyzed; 6) other features.

The report may include information about the testing facility, which information is relevant to the hospital, clinic, or laboratory in which data generation was conducted. Data generation can include measurements of the phase shifts at designated frequency ranges. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior episodes of similar tissue conditions, and any other characteristics of the tissue), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician). The report may include a sample data section, which may provide information about the tissue analyzed in the monitoring assessment. Report fields with this information can generally be populated using data entered by the user, some of which may be provided as pre-scripted selections (e.g., using a drop-down menu).

The report may include an assessment report section, which may include information generated after processing of the data as described herein. The interpretive report can include VEPS values associated with one or more reference samples. The interpretive report can include a characterization of the tissue condition. The interpretive report can include a diagnosis of a medical condition. The interpretive report can include, for example, the phase shifts at each frequency within a defined range (see, e.g., Table 2), the VEPS tissue signature (e.g. “beta: 1.2; gamma: 0.4”, or more simply, “1.2; 0.4”) and interpretation, i.e. characterization and diagnosis. The assessment portion of the report can optionally also include a recommendation(s) for treatment.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc. It will be readily appreciated that the report can include all or some of the elements above, with the proviso that the report generally includes at least the elements sufficient to provide the analysis requested by the user (e.g. tissue characterization, medical diagnosis).

### Devices and Systems

Also provided are devices and systems for practicing one or more of the above-described methods. The subject devices and systems thereof may vary greatly, and may include one or more of a digital synthesizer, transceiver, phase detector, data acquisition module, data processing module, and the like.

For example, devices of interest may comprise a transceiver, e.g. an induction coil array, e.g. a first induction coil and a second induction coil positioned opposite one another and configured such that a tissue placed between them will not touch the first induction coil or second induction coil; or an antennae array, i.e. first antenna and a second antenna positioned opposite one another and configured such that a tissue placed between them will not touch the first antenna or second antenna.

As another example, devices of interest may comprise a measurement system or phase detector operably linked—or capable of being operably linked—to the second induction coil of an induction coil array, e.g. as described above, where the measurement system is configured to measure a phase shift in one or more alternating currents between the induction coils of the array at two or more frequency ranges, e.g. a first frequency range and at a second frequency range; or a measurement system operably linked—or capable of being operably linked—to the second antenna of an antenna array, e.g. as described above, where the measurement system is configured to measure a phase shift in one or more voltages between the antennae of the array at two or more frequency ranges, e.g. at a first frequency range and at a second frequency range. In some instances, the first frequency range is between about 0.1 MHz and 150 MHz, e.g. between about 1 MHz and 70 MHz, e.g. between about 10 MHz and 60 MHz, between about 20 MHz and 50 MHz, between about 25 MHz and 40 MHz, e.g. between about 30 MHz and 35 MHz, i.e. about 33 MHz. In some instances, the second frequency range is between about 100 MHz and 500 MHz, e.g. between about 120 MHz and 200 MHz, e.g., between about 130 MHz and 190 MHz; between about 140 MHz and 180 MHz; between about 130 MHz and 190 MHz; between about 140 MHz and 180 MHz; between about 150 MHz and 170 MHz; e.g. between about 155 MHz and 165 MHz, i.e. about 160 MHz. Other relevant frequency ranges and frequencies, e.g., from 1 Hz to 1 THz, from 1 Hz to 20 GHz, from 10 KHz to 10 GHz, and the like, may be readily determined by the ordinarily skilled artisan, e.g. as known in the art or as described herein.

In some instances, the measurement system/phase detector is configured to determine the phase shift at a single time point. In some instances, the measurement system is further configured to determine changes in phase shift over time, i.e. at multiple time points, e.g. every 5 minutes, every 15 minutes, every 30 minutes, every 1 hour, every 2 hours, every 3 hours, every 4 hours, every day. In some instances, the measurement system is configured to transmit VEPS data by wireless communication.

As third example, devices of interest may comprise an analyzer element, e.g. a data acquisition module, a data processing module, etc., configured to calculate VEPS values from recorded phase shifts, to compare VEPS tissue signatures to a reference or panel of references, e.g. a panel of tissue classifiers, to determine the condition of the tissue, etc.

Similarly, systems of interest may comprise an induction coil array or an antenna array, e.g. configured as described above; and a measurement system operably linked to the second induction coil or second antenna of the array and configured to measure a phase shifts between the antennae or induction coils of the array, e.g. as described above. Systems of interest also include systems that comprise a measurement system capable of being operably linked to an induction coil array or antenna array, and configured to measure a phase shift between the antennae or induction coils of the array, e.g. as described above; and an analyzer element, e.g. a computer, etc., configured to compare a tissue signature to a reference or panel of references, e.g. as described above. In some instances, systems of interest comprise an induction coil array or an antenna array, e.g. configured as described above; a measurement system operably linked to the second induction coil or second antenna of the array and configured to measure a phase shifts between the antennae or induction coils of the array, e.g. as described above; and an analyzer element, e.g. a computer, etc., configured to compare a tissue signature to a reference or panel of references, e.g. as described above.

In addition to the above components, the subject devices and systems may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### Utility

The compositions, methods, devices and systems disclosed herein provide an advancement in the art for analyzing the health of a tissue in a subject. Prior to the discoveries disclosed here in, there existed no simple measurable criteria or parameter for the health of the normal human brain; e.g., like measuring blood pressure to determine the health and function of the cardiovascular system. The present disclosure demonstrates that VEPS or VEPS-like measurements of any kind (including, for example, combinations of amplitude and phase shift) taken at a single time point may serve as a simple-to-measure parameter of a healthy human tissue, e.g. human brain, and can be used to monitor normal human brain health as well as treatments that affect desirable targets of this kind—similar to the use of blood pressure measurements to determine the health of the human cardiovascular system. FIGS. 5, 6 and 7 illustrate examples of the different medical insight that can be obtained from different single frequency VEPS measurements and combinations of multiple frequency VEPS measurements for both diseased patients and healthy patients as parameters for identifying health, disease, and efficacy of medical treatment.

In view of the above, the disclosed compositions, methods, devices and systems find a number of uses in the art. These include, for example, in non-invasively determining the condition of a tissue, e.g. brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, trunk tissue, prostate tissue, breast tissue, esophagus tissue, GI tract tissue, etc., in an individual. Determinations of the condition of a tissue may be used in diagnosing, prognosing, and/or monitoring a host of medical conditions. The term “diagnosis” as used herein generally includes a prediction of a subject's susceptibility to a disease or disorder, a determination as to whether a subject is presently affected by a disease or disorder, classification of the subject's disease or disorder into a subtype of the disease or disorder (e.g. identification of a disease state or stage), a prognosis of a subject affected by a disease or disorder (e.g. likelihood that a patient will recover from the disease or disorder, prediction of a subject's responsiveness to treatment for the disease or disorder); and the monitoring a subject's condition to provide information as to the effect or efficacy of therapy. In some instances, the disclosed compositions, methods, devices and systems find particular use in providing a prognosis for a subject, e.g. the likelihood that a subject will recover from the disease or disorder, a prediction as to whether the subject will be responsive to a treatment, etc. In some instances, the disclosed compositions, methods, devices and systems find particular use in monitoring a tissue, e.g. during the development of a new therapy, or during the administration of a therapy.

For example, a number of different medical conditions are associated with abnormal tissue fluid content that is not discernible by the eye. The diagnosis, prognosis of these conditions is critical to their attenuation and treatment. Examples of such medical conditions include, but are not limited to: edema, hemorrhage, hematoma, ischemia, dehydration, the presence of a tumor, infection, brain degeneration, extravasation, internal bleeding, maternal hemorrhage, tissue health relative to age (e.g. premature aging of the tissue), and the like. The compositions, methods, devices, and systems of the present disclosure can be particularly useful in classifying the tissue as having one of these conditions.

Edema and Ischemia. Tissue edema is a pathological condition involving an increase in the amount of fluid in tissue. The accumulation of fluid can be extracellular, intracellular or both. Extracellular edema is caused either by increased ultrafiltration or decrease in reabsorption. Intracellular edema can be caused by ischemia and the resulting intracellular hyperosmolarity or as a consequence of extracellular hypotonicity. Independent of the edema type, the condition is one in which the amount of liquid in the tissue increases and the balance is changed, usually as a function of time after an event has occurred. Tissue edema is of substantial concern when it occurs in the brain or in the lung. In the brain, extracellular edema develops in a delayed fashion, over a period of hours or days, after a large hemispheric stroke and is a cause of substantial mortality. Ischemic brain edema begins with an increase in tissue Na+ and water content and continues with blood brain barrier breakdown and infarction of both the parenchyma and the vasculature itself.

A study of the Center for Disease Control and Prevention for the period from 1995 to 2001 indicates that at least 1.4 million annual traumatic brain injuries occur in the USA alone. These resulted in about 1.1 million emergency department visits, 235,000 hospitalizations and about 50,000 deaths. About 1,100 incidents per 100,000 in population occur in the age group from 0 to 4 years. Head injury causes more deaths and disability than any other neurological condition under the age of 50 and occurs in more than 70% of accidents. It is the leading cause of death in males under 35 yr old. Fatalities may not result from the immediate injury; rather, progressive damage to brain tissue develops over time. In response to trauma, changes occur in the brain that requires monitoring to prevent further damage.

Brain swelling can be caused by an increase in the amount of blood to the brain. Brain edema is one of the most important factors leading to morbidity and mortality in brain tumors. Cerebral edema, which is an increase in brain volume caused by an absolute increase in tissue water content, ensues. The accumulation of fluid can be extracellular, intracellular or both. Vasogenic edema results from trans-vascular leakage often caused by the mechanical failure of the tight endothelial junction of the blood-brain barrier and increased ultrafiltration or decrease in re-absorption. Vasogenic edema also results from extravasation of protein rich filtrate in interstitial space and accumulation of extracellular fluid. Cytotoxic edema is characterized by cell swelling. Cytotoxic edema is an intracellular process resulting from membrane ionic pump failure. It is very common after head injury and it is often associated with post-traumatic ischemia and tissue hypoxia. The primary mechanism is reduction of sodium-potassium ATPase pump efficiency due to local hypoxia and ischemia. This type of edema occurs in cancer with compression of microcirculation. Interstitial or hydrocephalic edema occurs when there is an accumulation of extracellular fluid in the setting of hydrocephalus. Intraventricular tumors or tumors that constrict ventricles can cause this type of edema.

Independent of the edema type, the condition is one in which the amount of liquid in the tissue increases or the balance is changed. Edema is of substantial concern when it occurs in the brain. The characteristics of brain edema, is that it develops in a delayed fashion, over a period of hours or days, after the brain trauma has occurred and is a cause of substantial mortality. Detection and continuous monitoring of edema in the brain is essential for assessment of medical condition and treatment.

Pulmonary edema is often associated, with lung injury and also requires continuous monitoring and treatment. Detection and continuous monitoring of edema in the brain and lung is useful for assessment of medical condition and treatment.

Ischemia of tissues and organs is caused by a change in normative physiological conditions such as deprivation of oxygen and blood flow. It can occur inside the body, for instance as a consequence of impediments in blood flow. Ischemia also can occur outside the body when organs preserved for transplantation are transported. Ischemia results in changes in the intracellular composition which is accommodated by changes in the water content properties of the intracellular and extracellular space and leads to cell death.

Therefore, in medical applications it is important to be able to detect changes in water content properties which are indicative of the occurrence of edema and ischemia.

Internal and Interperitoneal Bleeding. Trauma is the third most common cause of death in all age groups and the leading cause of death in the first three decades of life. Of all traumatic injuries abdominal and pelvic injuries contribute to about 20% of the fatalities. In addition, death from abdominal hemorrhage is a common cause of preventable death in trauma patients. Bleeding is the cause of one in four maternal deaths worldwide. Death may occur in less than two hours after the onset of bleeding associated with childbirth. In addition to trauma, abdominal bleeding also occurs in several post-surgery conditions. Unfortunately, early intraperitoneal bleeding cannot be detected by vital signs (rate pulse or blood pressure) and it becomes evident only after a critical amount of blood has found its way into the abdominal cavity. Therefore, death from abdominal hemorrhage is a common cause of preventable death in trauma patients. However, early detection of intraperitoneal bleeding may play a critical role in the patient survival.

Extravasation. Extravasation is the unwanted passage or escape of blood, serum, lymph or therapeutic drugs directly into body tissues. Signs and symptoms may include the sudden onset of localized pain at an injection site, sudden redness or extreme pallor at an injection site, or loss of blood return in an intravenous needle. Extravasation can lead to skin and tissue necrosis, and “Compartment Syndrome” (a pathologic condition caused by the progressive development of arterial compression and reduction of blood supply).

Similar to the medical conditions described above, extravasation results in a change in water content properties in the tissue (typically at or near an injection site). Thus, it would be desirable to detect extravasation (preferably by an on-contact system).

Tissue aging and aging treatment target. As tissues age, stereotypical structural, chemical and functional changes occur. In certain instances, the changes may occur prematurely, resulting in “premature aging”, or “pathological aging”, of the tissue.

For example, in brain tissue, stereotypical structural and neurophysiological changes occur, accompanied in some individuals by cognitive decline. Computed Tomography (CT) studies have found that the cerebral ventricles expand as a function of age in a process known as ventriculomegaly. MRI studies have reported age-related regional decreases in cerebral volume (Craik, F. et al. (2000). The Handbook of Aging and Cognition (2nd ed.). Mahwah, N.J.: Lawrence Erlbaum; Raz, N. et al. (2005). Regional Brain Changes in Aging Healthy Adults: General Trends, Individual Differences and Modifiers. Cereb. Cortex 15 (11): 1676-1689). Studies using Voxel-based morphometry have identified areas such as the insula and superior parietal gyri as being especially vulnerable to age-related losses in grey matter of older adults (Henkenius, A. et al. (2003). “Mapping cortical change across the human life span”. Nature Neuroscience 6 (3): 309-315). Also vulnerable are anterior language cortices, responsible for certain language functions such as word retrieval and production. On the other hand, areas such as the cingulate gyrus and occipital cortex surrounding the calcarine sulcus appear exempt from this decrease in grey matter density over time (Henkenius, A. et al., supra).

This loss in grey matter in the brain is associated at least in part with a loss of synapses between neurons. See, e.g. US Application No. US2012/328601, the disclosure of which is incorporated herein by reference. Synapse loss begins at at least about age 20, and may or may not be accompanied by cognitive decline. Typically, if cognitive decline occurs, it is a modest disruption of memory often referred to as “age-associated cognitive impairment” or “mild cognitive impairment” (MCI) that manifests as problems with memory or other mental functions such as planning, following instructions, or making decisions that have worsened over time while overall mental function and daily activities are not impaired. Thus, although significant neuronal death may not typically occur, neurons in the aging brain are vulnerable to sub-lethal age-related alterations in structure, synaptic integrity, and molecular processing at the synapse, all of which impair cognitive function.

Another hallmark structural change that occurs in the aging brain is the development of neurofibrillary tangles. Neurofibrillary tangles develop in both normal aging and aging-associated neuro-pathologies (e.g., Alzheimer's disease, Parkinson's disease, diabetes, hypertension and arteriosclerosis). However, in contrast to aging-associated neuro-pathologies, during normal aging of the brain, there is a general increase in the density of tangles with no significant difference in where tangles are found.

Change in the synthesis of neurotransmitters and neurotransmitter receptors are also observed in the aging brain. For example, studies using positron emission tomography (PET) in living human subjects have shown a significant age-related decline in dopamine synthesis (Hof, P. R. et al. (2009). Handbook of the neuroscience of aging. London: Elsevier), notably in the striatum and extrastriatal regions (excluding the midbrain) (Ota, M et al. (2006). “Age-related decline of dopamine synthesis in the living human brain measured by positron emission tomography with Lo-[β-11C]DOPA”. Life Sciences 79 (8): 730-736). Significant age-related decreases in dopamine receptors D1, D2, and D3 have also been reported (Kaasinen, V. et al. (2000). “Age-related dopamine D2/D3 receptor loss in extrastriatal regions of the human brain”. Neurobiology of Aging 21 (5): 683-688; Wang, Y. et al. (1998). “Age-Dependent Decline of Dopamine D1 Receptors in Human Brain: A PET Study”. Synapse 30 (1): 56-61). Decreasing levels of different serotonin receptors and the serotonin transporter, 5-HTT, have also been shown to occur with age. Studies conducted using PET methods on humans, in vivo, show that levels of the S2 receptor in the caudate nucleus, putamen, and frontal cerebral cortex, decline with age (Wong, D. F., et al. (1984). “Effects of age on dopamine and serotonin receptors measured by positron tomography in the living human brain”. Science 226 (4681): 1393-1396).

Stereotypical structural, chemical and functional changes accompany aging in other tissues as well. For example, in the aging respiratory system, lung elasticity decreases, stiffness of the chest wall increases, and respiratory muscle strength declines. These alterations contribute to gradual, but progressive, reductions in forced vital capacity, expiratory flow rates, diffusing capacity, gas exchange, ventilatory drive, and respiratory sensation as the individual ages. In the circulatory system, increasing age is associated with increased intimal thickness, vascular smooth muscle hypertrophy, fragmentation of the internal elastic membrane and an increase in the amount of collagen and collagen cross-linking in arterial walls. Stiffening of the arterial tree alters afterload and left ventricular geometry in the heart, and although resting left ventricular systolic function is maintained, left ventricular diastolic function changes substantially, which may lead to the development of left ventricular hypertrophy. In the aging liver, a decline in tissue volume and blood flow is observed, resulting in a decreased metabolic rate and rate of drug clearance.

Other examples of conditions associated with tissue aging will be known to the ordinarily skilled artisan. Early detection of premature onset of such conditions, e.g., as described above or as known in the art, will play a critical role in the long-term health and survival of individuals.

Obtaining a VEPS-based tissue signature may be used to diagnose a medical condition, or identify a tissue condition that requires further observation, e.g. medical imaging. In some instances, the VEPS signature may be used alone to provide a diagnosis, a prognosis, to monitor a treatment, etc. In some instances, the VEPS signature may be employed in combination with other clinical parameters, e.g. age, weight, overall health, risk factors for the disease or disorder, etc. as known in the art, to provide the diagnosis, the prognosis, monitor responsiveness to a treatment, etc. As such, in some embodiments, the method further comprises determining a clinical parameter, and providing a determination of a condition of a tissue in a subject based on a VEPS signature and the clinical parameter.

It is obvious that the various measurements listed above are expensive and not as convenient to use as, for instance, blood pressure measurements as a target for the cardiovascular system, statoscope measurements for the lung, or even ECG measurements for the heart. In contrast, VEPS and VEPS-like technology is inexpensive, simple to perform, and provides a wealth of information that may be used to diagnose a subject, provide a prognosis, monitor treatment, or monitor tissue health during drug discovery. The examples presented herein illustrate the different medical insight that can be obtained from different single frequency VEPS measurements and combinations of multiple frequency VEPS measurements for both diseased patients and healthy patients as targets for identifying disease, health, and efficacy of medical treatment.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Example 1

### Materials and Methods

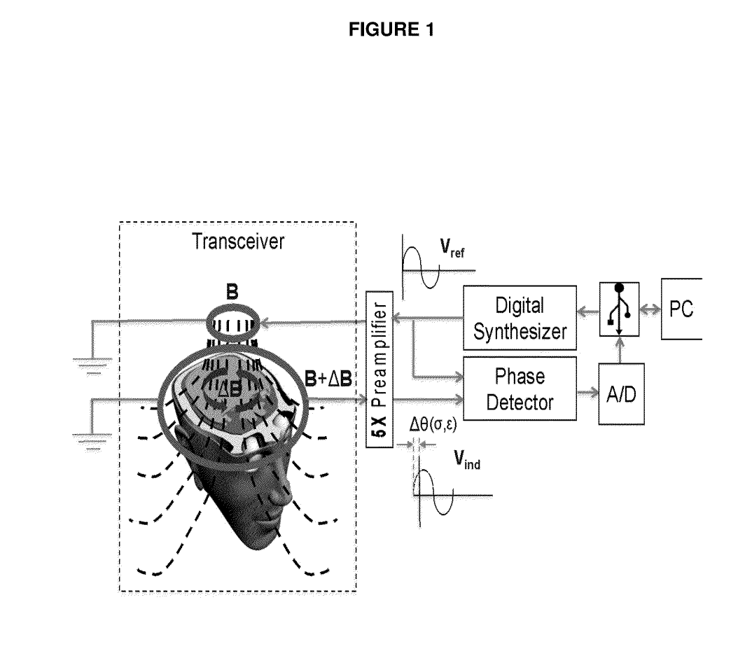

Biophysical Considerations for Inductive Phase Shift Measurements. A schematic of the human head/coil geometrical configuration used in this study is shown in FIG. 1. The device is very simple. It consists of two coupled coils of different radii in an inductor-sensor arrangement. The coils are coaxially centered. The brain (head) is placed between the coils. An alternating current, lejwt is injected into the inductor coil. The current generates a primary magnetic field B that is detected by the sensor coil. The volume of tissue confined between the coils produces a perturbation of the primary magnetic field, (AB). The perturbation is a function of the complex impedance of the brain tissue in the volume between the coils. The perturbation is evaluated by comparing the field in the sensor coil B+ΔB, to the primary field B. Changes in the magnetic field represent volumetric changes in the brain composition complex impedance. A robust way to detect changes in the magnetic field is to measure the phase shift between the inductor coil and the sensor coil. Measuring the phase shift as a function of the injected current frequency produces “volumetric electromagnetic phase-shift spectroscopy” (VEPS) data. A simple way to measure the phase shift is through a “voltage relative to voltage” arrangement (Mori, K., Met al. (2002), “Temporal profile of changes in brain tissue extracellular space and extracellular ion (Na+, K+) concentrations after cerebral ischemia and the effects of mild cerebral hypothermia.” Journal of neurotrauma 19(10): 1261-1270; Schwan, H. P. (1957), “Electrical properties of tissue and cell suspensions”, Adv. Biol. Med. Phys., 5:147-209). In this arrangement the frequency dependent phase difference between the voltage in the inductor coil and the voltage in the sensor coil are used to estimate the VEPS.

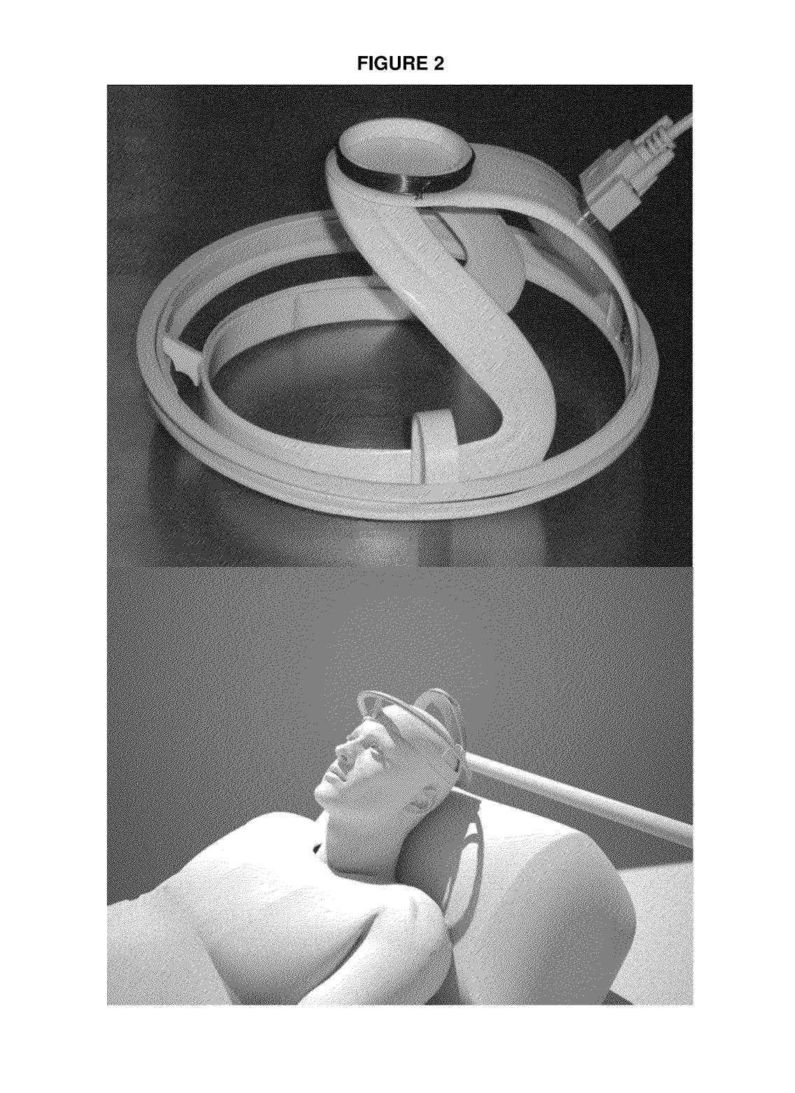

Experimental VEPS Prototype. Following is a brief description of the VEPS data acquisition device. The system consists of five modules: digital synthesizer, transceiver, phase detector, data acquisition and data processing. The modules are shown in a block diagram in FIG. 1. The digital synthesizer is a signal generator AD9958 (Analog Device Inc. Norwood, Mass., USA). It supplies a sinusoidal current, I cos(ωt), of approximately 10 mA rms in the frequency range of 1-200 MHz. The current is supplied at 200 pre-programmed equally spaced frequencies, under PC control. The transceiver consists of two concentric coils with radii of R1=3.2 cm and R2=11 cm, separated by a distance of 10 cm. Both coils were built from ten turns of magnet wire AWG22 rolled on an ergonomic plastic harness specifically designed for an adult human head (FIG. 2). The coil inductances, calculated from Faraday's law, are approximately 67.4 and 796.4 μH for the inductor and sensor coils, respectively. The estimated mutual inductance coefficient is approximately M=72.8 μH. To avoid inductive pickup the leads of the coils are twisted. A commercial device, AD8302 (Analog Devices Inc. Norwood, Mass., USA) was used for phase detection. The AD8302 is a fully integrated RF IC for measuring differences in phase between two signals with a resolution of 10 mV/degree. The signals from the inductor and sensor coils are connected through a 5X preamplifier SR445 (Standford Research System Inc. Sunnyvale, Calif., USA) to the digital synthesizer and phase detector module, as shown in FIG. 1. The data acquisition (A/D) module uses a 10-Bit Analog-to-Digital module microcontroller 18F4550 (Microchip Technology Inc., Chandler, Ariz., USA). The VEPS data at each frequency is the average from 1024 measurements at that frequency. The sensor sample rate is 48 kSamples/sec. Photographs of the clinical VEPS inductor-sensor prototype and the way it was positioned on the head of a brain damage patient in the Critical Care Unit (CCU) are shown in FIG. 2.

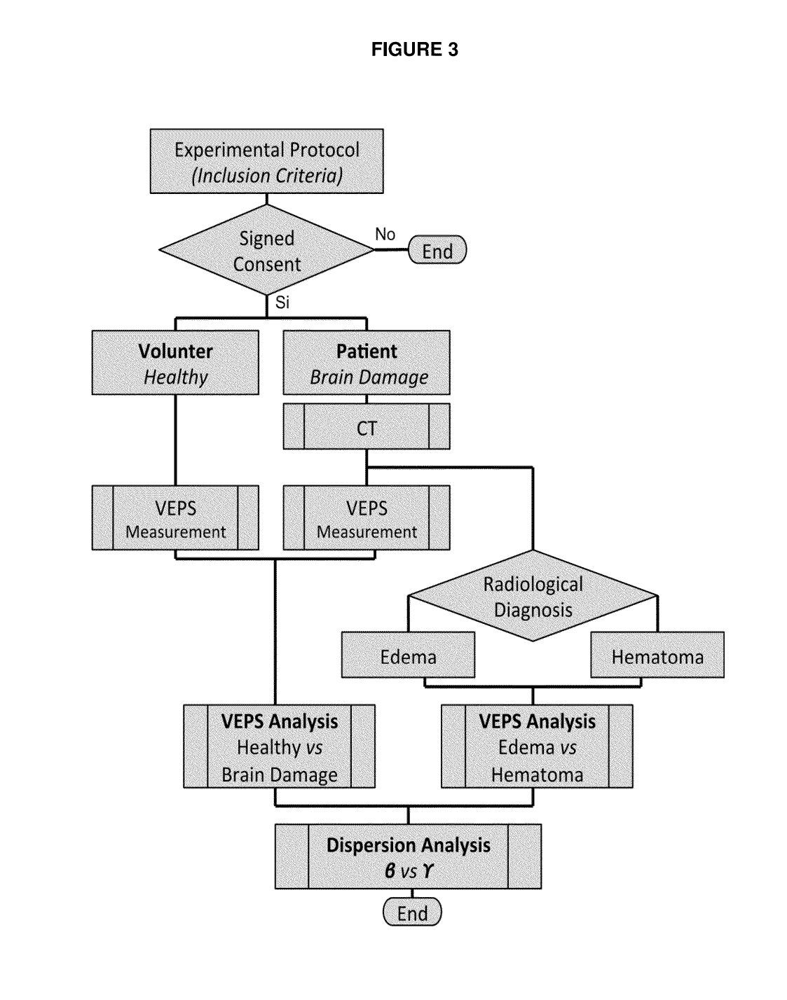

Experimental Design. The inclusion criteria were: female and males in the age range of from 18 to 70 years old without metallic prostheses or pacemakers. FIG. 3 shows a flow diagram of the study. The study consists of acquiring non-invasive VEPS data from two groups of subjects: a) Healthy volunteers (46 volunteers, age 18 to 48) and b) Patients with brain damage admitted to the CCU as a result of one of the following pathologies: neuroinfection, brain vascular event or craneoencefalic trauma (8 patients, ages 27 to 70). The patients with brain damage were further classified into two typical clinical conditions with regards to the genesis of the pathology: a) Edema—diffuse or localized edema without hemorrhage, and b) Hematoma—epidural, subdural, parenchymal or subarachnoid well localized hematomas. Although hematomas are associated with edema, for simplicity we have chosen to call the condition brain-injury+hematoma, “hematoma”, because the predominant pathology of accumulation of blood. The neuroradiology department evaluated the brain pathology of the patients with computerized tomography (CT), before the VEPS study. In both, healthy volunteers and patients we measured: a) the Craneoencefalic Perimeter (CP) with a common 1 mm resolution tape and b) the VEPS in the range of 1 to 200 MHz at 200 pre-programmed frequencies (equally spaced) with the prototype described earlier. The VEPS data was normalized with respect to CP to minimize the intrinsic head volume effect on the VEPS measurements. The VEPS/CP data from healthy volunteers was compared with the data from patients with brain damage. Among patients with brain damage the VEPS/CP data was compared between those diagnosed with edema and those diagnosed with “hematoma”. Because of the relatively small number of samples, a non-parametric statistical Mann-Whitney U test was applied to the multifrequency VEPS/CP data analysis. The statistical analysis employed the program STATISTICA V7.0 (Stat Soft. Inc) and the significance level criteria is p<0.05.

### Results

The study reported here was done with 46 healthy volunteers (ages 18 to 48) and eight patients with brain damage (ages 27 to 70). A listing of the subjects relevant personal data and their Craneoencefalic Perimeter (CP) [cm] is given in Table 1.

TABLE 1
Listing of data for the healthy volunteers and
brain damage patients enrolled in the study.

| Group | Condition | Number | Sex | Age (yrs) | CP (cm) |
|---|---|---|---|---|---|
| Healthy | Young | 1 | Male | 31 | 58 |
| | | 2 | Male | 30 | 54 |
| | | 3 | Male | 27 | 57 |
| | | 4 | Male | 20 | 55.5 |
| | | 5 | Male | 20 | 55 |
| | | 6 | Male | 20 | 55.5 |
| | | 7 | Male | 30 | 58 |
| | | 8 | Male | 27 | 57 |
| | | 9 | Male | 22 | 56 |
| | | 10 | Male | 23 | 57 |
| | | 11 | Male | 24 | 56 |
| | | 12 | Male | 18 | 57 |
| | | 13 | Male | 20 | 55.5 |
| | | 14 | Male | 18 | 56 |
| | | 15 | Male | 19 | 55.5 |
| | | 16 | Male | 31 | 55.5 |
| | | 17 | Male | 20 | 57.5 |
| | | 18 | Male | 30 | 57 |
| | | 19 | Female | 22 | 57 |
| | | 20 | Female | 26 | 54.5 |
| | | 21 | Female | 19 | 56 |
| | | 22 | Female | 20 | 54.4 |
| | | 23 | Female | 22 | 56 |
| | | 24 | Female | 22 | 57 |
| | | 25 | Female | 22 | 55 |
| | | 26 | Female | 25 | 53 |
| | | 27 | Female | 19 | 57.3 |
| | | 28 | Female | 19 | 58 |
| | | 29 | Female | 20 | 55 |
| | | 30 | Female | 20 | 56 |
| | | 31 | Female | 18 | 56 |
| | | 32 | Female | 19 | 56 |
| | | 33 | Female | 21 | 58 |
| | | 34 | Female | 21 | 57 |
| | | 35 | Female | 21 | 54 |
| | | 36 | Female | 25 | 56 |
| | | 37 | Female | 24 | 54.5 |
| | | 38 | Female | 22 | 57 |
| | Adult | 39 | Male | 48 | 57 |
| | | 40 | Female | 40 | 56 |
| | | 41 | Male | 46 | 57 |
| | | 42 | Female | 46 | 54 |
| | | 43 | Male | 42 | 55 |
| | | 44 | Male | 40 | 55 |
| | | 45 | Female | 48 | 54 |
| | | 46 | Female | 40 | 55 |
| Brain | Edema | 47 | Female | 61 | 53 |
| Damage | | 48 | Male | 48 | 56 |
| | | 49 | Female | 27 | 53.5 |
| | | 50 | Male | 27 | 56 |
| | Hematoma | 51 | Male | 70 | 56.5 |
| | | 52 | Male | 30 | 56.5 |
| | | 53 | Female | 58 | 57 |
| | | 54 | Female | 27 | 55 |

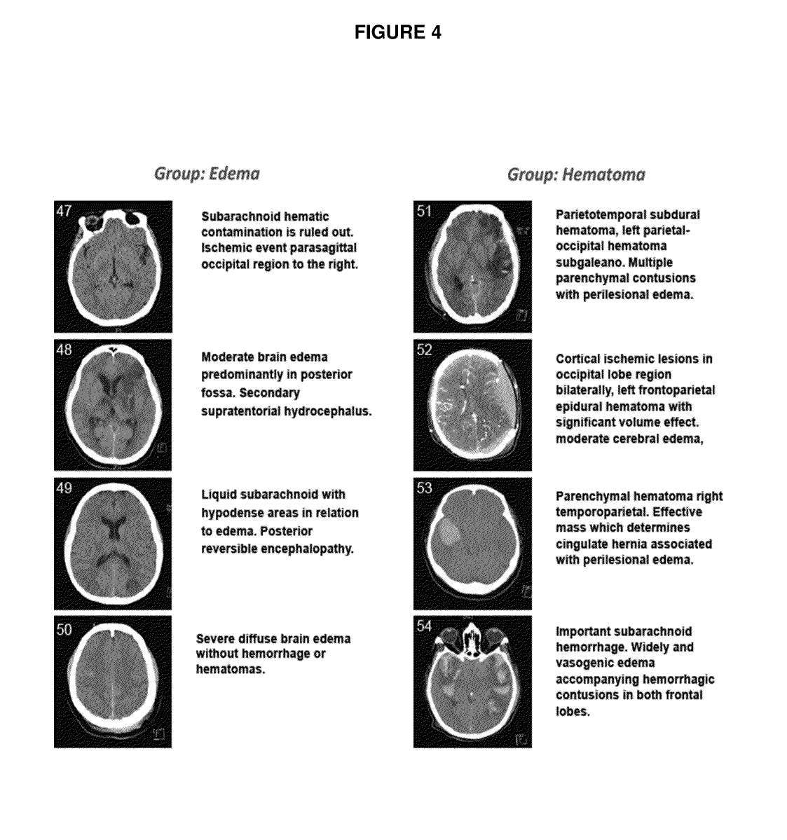

Multifrequency VEPS measurements were acquired with the specially build VEPS device described in the “material and methods” section and shown in FIGS. 1 and 2. The VEPS data from patients with brain injury was correlated with computerized tomography (CT) images of the head using the experimental protocol in the flow diagram in FIG. 3. FIG. 4 shows CT's of the brain damaged patients head, divided into two groups according to their pathologies: edema or hematoma. The clinical neurological evaluation is given next to each CT image. The CT images on left (edema) show moderate to severe diffuse brain edema without hemorrhage or hematomas. Epidural, subdural, parenchymal or subarachnoid well-localized hematomas are seen in the images on right (hematoma).

As indicated earlier, because of the relatively small number of subjects, the non-parametric statistical Mann-Whitney U test (STATISTICA V7.0 (Stat Soft. Inc) was applied to the multifrequency VEPS/CP data analysis. The highlight of the analysis is displayed in Table 2. The non-parametric statistical Mann-Whitney U test detected statistically significant differences between the various VEPS measurements in healthy and brain-damaged subjects, with a significance level of P<0.05, in the frequency ranges from 26 MHz to 39 MHz and from 153 MHz to 166 MHz. In the frequency range from 26 MHz to 39 MHz there is astatistically significant difference between VEPS/CP in healthy volunteers and patients with brain damage. In the frequency range from 153 MHz to 166 MHz, the non-parametric statistical Mann-Whitney U test, which is designed for small number of data points, indicates statistically significant difference between the VEPS/CP measurement in patients with brain edema and those with brain hematoma.

To display the results of the measurements in a concise form we calculated for each subject two parameters, β and γ. The two parameters, β and γ, are the sum of all the values of VEPS/CP [degrees/cm] in the ranges of frequencies, from 26 MHz to 39 MHz and from 153 MHz to 166 MHz, at the specific frequencies listed in Table 2, respectively.

FIG. 5 shows the β value for all the subjects of this study as a function of the subject age. It shows that in healthy individuals there is a strong correlation between the β value and age (R2=0.6299), but that in brain diseased patients there is no correlation with age (R2=1.9E-5). There is, however, a significant statistical difference between the β value of healthy volunteers and those with a brain condition as also determined from Table 2. It is interesting to note that the β value versus age curve for healthy individuals intersects with that for a pathological brain condition of edema or hematoma at an age of about 77. This suggests that the measurements of the β value alone can detect brain damage effectively in young subjects, but that it will fails for older patients. FIG. 6 shows the γ value for all the subjects of this study as a function of age. It shows that in healthy individuals there is a correlation between the γ value and age (R2=0.2162), but that in brain diseased patients there is no correlation with age. Furthermore, there does not seem to be a distinction between healthy and diseased brains with age. However, as table 2 and FIG. 6 indicates, there is a statistically significant difference between patients with hematoma and edema. It is interesting to notice that the correlations of the β and γ parameters with age have a different sign slope for β and γ.

Tables 2 and FIGS. 5 and 6 show that the diagnostic of the condition of the brain is a function of two VEPS parameters in the β and γ ranges of frequency. This suggested to us that a display of the data for each individual in the multifrequency classifier modality shown in FIG. 7 might have diagnostic value. FIG. 7 shows the β and γ parameters for each individual in the study, represented as a data point. Each data point in the figure is identified with the subject number in Table 1. It is evident that in the representation of FIG. 7, patients with a brain condition stand out from the healthy volunteers and the disease modality of edema is separated from hematoma. FIG. 7 bears the hallmark of a scalar classifier display.

TABLE 2
Statistical analysis with a Mann-Whitney U test of the VEPS/CP
(degrees/cm) data for the experimental groups and subgroups in ranges
of frequencies in which a statistically significant difference of P < 0.05
between them, was found.

| | | | | | | | | |
|---|---|---|---|---|---|---|---|---|
| | Freq | Rank | Rank | | | | Valid | Valid |
| Bandwidth | (MHz) | Sum | Sum | U | Z | p-level | N | N |

Healthy vs Brain damage

| | | | | | | | | |
|---|---|---|---|---|---|---|---|---|
| β | 26 | 1431 | 54 | 18 | 4.0420 | 0.00005 | 46 | 8 |
| | 27 | 1444 | 41 | 5 | 4.3585 | 0.00001 | 46 | 8 |
| | 28 | 1398 | 87 | 51 | 3.2384 | 0.00120 | 46 | 8 |
| | 29 | 1443 | 42 | 6 | 4.3342 | 0.00001 | 46 | 8 |
| | 30 | 1388 | 43 | 7 | 4.2982 | 0.00002 | 45 | 8 |
| | 31 | 1375 | 110 | 74 | 2.6784 | 0.00740 | 46 | 8 |
| | 32 | 1386 | 99 | 63 | 2.9463 | 0.00322 | 46 | 8 |
| | 33 | 1410 | 75 | 39 | 3.5306 | 0.00041 | 46 | 8 |
| | 34 | 1423 | 62 | 26 | 3.8472 | 0.00012 | 46 | 8 |
| | 35 | 1441 | 44 | 8 | 4.2855 | 0.00002 | 46 | 8 |
| | 36 | 1449 | 36 | 0 | 4.4803 | 0.00001 | 46 | 8 |
| | 37 | 1449 | 36 | 0 | 4.4803 | 0.00001 | 46 | 8 |
| | 38 | 1433 | 52 | 16 | 4.0907 | 0.00004 | 46 | 8 |
| | 39 | 1419 | 66 | 30 | 3.7498 | 0.00018 | 46 | 8 |

Edema vs Hematoma

| | | | | | | | | |
|---|---|---|---|---|---|---|---|---|
| γ | 153 | 11 | 25 | 1 | −2.0207 | 0.04330 | 4 | 4 |
| | 154 | 10 | 26 | 0 | −2.3094 | 0.02092 | 4 | 4 |
| | 155 | 11 | 25 | 1 | −2.0207 | 0.04330 | 4 | 4 |
| | 156 | 11 | 25 | 1 | −2.0207 | 0.04330 | 4 | 4 |
| | 161 | 10 | 26 | 0 | −2.3094 | 0.02092 | 4 | 4 |
| | 162 | 10 | 26 | 0 | −2.3094 | 0.02092 | 4 | 4 |
| | 163 | 10 | 26 | 0 | −2.3094 | 0.02092 | 4 | 4 |
| | 164 | 10 | 26 | 0 | −2.3094 | 0.02092 | 4 | 4 |
| | 165 | 10 | 26 | 0 | −2.3094 | 0.02092 | 4 | 4 |
| | 166 | 11 | 25 | 1 | −2.0207 | 0.04330 | 4 | 4 |

### Discussion

The complex impedance of biological tissue displays, in the range of frequency, from DC to GHz, has three distinctive dispersions (Grimnes S., et al. “Bioimpedance and Bioelectricity Basics” (2000). Academic Press USA). The electrical permittivity and conductivity of the three main dielectric dispersions have been labeled α, β, and γ. They occur at increasing frequencies from DC through MHz to GHz, respectively. The α-dispersion is caused by the relaxation in the counter-ion atmosphere surrounding the charged cell membrane surface, the β-dispersion is produced by Maxwell-Wagner relaxation, an interfacial relaxation process occurring in materials containing boundaries between two different dielectrics and the γ-dispersion by the relaxation of free water within tissues (Schurer, L., et al. “Is postischaemic water accumulation related to delayed postischaemic hypoperfusion in rat brain?” (1998). Acta Neurochirurgica 94(3-4): 150-154).

Measurement of the spectral characteristics of biological tissue provides information on the structure and changes in composition of biological tissues, in particular the ratio of intracellular to extracellular fluids. The use of bioelectrical impedance measurements to detect water content and edema in the body was suggested already half a century ago (Morucci, J. P., et al. “Bioelectrical impedance techniques in medicine” (1996). Critical Reviews in Biomedical Engineering 24(4-6): 655-677; Nierman, D. M., et al. “Transthoracic bioimpedance can measure extravascular lung water in acute lung injury” (1996). J Surg Res. 65(2): 101-8; Grasso, G., et al. “Assessment of human brain water content by cerebral bioelectrical impedance analysis: A new technique and its application to cerebral pathological conditions” (2002). Neurosurgery 50(5): 1064-1072). Bioelectric measurements have evolved into an imaging technology known as Electrical Impedance Tomography (EIT) that uses arrays of contact electrodes to inject sub-sensory currents in the body and measure voltage to produce a map of electric impedance of tissue for use in various medical imaging applications, including detection of edema (Henderson, R. P., et al. “Impedance camera for spatially specific measurements of thorax” (1978). IEEE Trans. Biomed. Eng. 25(3): 250-254; Webster, J. G., Electrical Impedance Tomography, New York: Adam Hilger, 1990; Metherall, P., et al. “Three-dimensional electrical impedance tomography” (1996). Nature 380: 509-512; Newell, J. C., et al. “Assessment of acute pulmonary edema in dogs by electrical impedance imaging” (1996). IEEE Trans Biomed Eng 43(2): 133-8; Otten, D. M., et al. “Cryosurgical monitoring using bio-impedance measurements—a feasibility study for electrical impedance tomography” (2000). IEEE—Trans of Biomedical Eng 27(10): 1376-1382; Lionheart, W. R. “EIT reconstruction algorithms: pitfalls, challenges and recent developments” (2004). Physiol Meas 25: 125-142; Holder, D. S. “Electrical impedance tomography: methods, history and applications” (2005). London: IOP Publishing Ltd 456; Tang, T., et al. “Quantification of intraventricular hemorrhage with electrical impedance tomography using a spherical model” (2011). Physiol. Meas. 32(7): 811-21). Bioelectrical measurements by magnetic induction with non-contact electrical coils are considered a valuable alternative to contact electrode measurement (Tarjan, F. P., et al. “Electrodeless measurements of the effective resistivity of the human torso and head by magnetic induction” (1968). IEEE Trans. Biomed. Eng. 15: 266-78; Netz J., et al. “Contactless impedance measurement by magnetic induction—a possible method for investigation of brain impedance” (1993). Physiol. Meas. 14: 463-71; Griffiths H., et al. “Magnetic induction tomography—a measuring system for biological materials” (1999). Ann. NY Acad. Sci. 873: 335-45; Al-Zeiback, A., et al. “A feasability study of in vivo electromagnetic imaging” (1993). Phys. Med. Biol. 38:151-160; Korjenevsky, A. V., et al. “Progress in Realization of Magnetic Induction Tomography” (1999). Ann NY Acad Sci. 873: 346-352; Griffiths, H. “Magnetic Induction tomography” (2001.) Meas. Sci. Technol. 12: 1126-31; Scharfetter, H., et al. “Magnetic induction tomography: Hardware for multi-frequency measurements in biological tissues” (2001). Physiol Meas. 22(1): 131-146; Soleimani, M., et al. “Absolute Conductivity Reconstruction in Magnetic Induction Tomography Using a Nonlinear Method” (2006). IEEE Trans Medical Imaging 25(12): 1521-1530; Hart, L. W., et al. “A noninvasive electromagnetic conductivity sensor for biomedical applications” (1988). IEEE Trans. Biomed. Eng. 35: 1011-22; Merwa, R., et al. “Detection of brain oedema using magnetic induction tomography: a feasability study of likely sensitivity and detectability” (2004). Physiol. Meas. 25: 347-57; Kao, H. P., et al. “Correlation of permittivity and water content during cerebral edema” (1999). IEEE Trans. Biomed. Eng. 46: 1121-8; Scharfetter, H., et al. “Biological tissue characterization by magnetic induction spectroscopy (MIS): requirements and limitations” (2003). IEEE Trans. Biomed. Eng. 50: 870-80). Inductive measurement does not require galvanic coupling between the electrode and the skin or the tissue under measurement. In the particular case of brain conductivity measurement for edema detection, the skull does not represent a barrier for the magnetic field (Tarjan, F. P., et al. “Electrodeless measurements of the effective resistivity of the human torso and head by magnetic induction” (1968). IEEE Trans. Biomed. Eng. 15: 266-78; Netz J., et al. “Contactless impedance measurement by magnetic induction—a possible method for investigation of brain impedance” (1993). Physiol. Meas. 14: 463-71). This is why we have chosen non-contact electromagnetic measurements for our technology. Non-contact measurements have found applications in developing an alternative technique for electrical imaging of tissue—Magnetic Induction Tomography (MIT) and its different variants (Griffiths H., et al. “Magnetic induction tomography—a measuring system for biological materials” (1999). Ann. NY Acad. Sci. 873: 335-45; Al-Zeiback, A., et al. “A feasability study of in vivo electromagnetic imaging” (1993). Phys. Med. Biol. 38:151-160; Korjenevsky, A. V., et al. “Progress in Realization of Magnetic Induction Tomography” (1999). Ann NY Acad Sci. 873: 346-352; Griffiths, H. “Magnetic Induction tomography” (2001.) Meas. Sci. Technol. 12: 1126-31; Scharfetter, H., et al. “Magnetic induction tomography: Hardware for multi-frequency measurements in biological tissues” (2001). Physiol Meas. 22(1): 131-146; Soleimani, M., et al. “Absolute Conductivity Reconstruction in Magnetic Induction Tomography Using a Nonlinear Method” (2006). IEEE Trans Medical Imaging 25(12): 1521-1530). Non-contact measurements have been considered for detecting shift of water content in tissue and edema through both spectroscopy and imaging (Hart, L. W., et al. “A noninvasive electromagnetic conductivity sensor for biomedical applications” (1988). IEEE Trans. Biomed. Eng. 35: 1011-22; Merwa, R., et al. “Detection of brain oedema using magnetic induction tomography: a feasability study of likely sensitivity and detectability” (2004). Physiol. Meas. 25: 347-57; Kao, H. P., et al. “Correlation of permittivity and water content during cerebral edema” (1999). IEEE Trans. Biomed. Eng. 46: 1121-8; Scharfetter, H., et al. “Biological tissue characterization by magnetic induction spectroscopy (MIS): requirements and limitations” (2003). IEEE Trans. Biomed. Eng. 50: 870-80). The VEPS technology that we have developed is based on the wealth of biophysical and bioengineering work from decades of previous research in the field. The novelty of our work is the concept of measuring the electromagnetic phase-shift from a composite volume of tissue in a range of relevant frequencies (U.S. Pat. No. 7,638,341; U.S. Pat. No. 7,910,374; U.S. Pat. No. 8,101,421). This leads to a very simple, inexpensive and robust device that produces spectral electromagnetic data that lend themselves to analysis with classifier technology rather than imaging. This technology will help address the problem of a lack of health services and access to medical imaging facilities faced by the many around the world.

The significance of the data gathered in this experiment is best understood through Table 3 (below).

TABLE 3
Electrical conductivity (S/m) at specific frequencies for brain
tissue, human serum and blood. From Stoy, R. D., et al. (1982)
Dielectric properties of mammalian tissues from 0.1 to 100 MHz;
a summary of recent data. Phys. Med. Biol. 27(4): 501-513; Duck,
F. A “Physical Properties of Tissue” London: Academic
(1990); Gabriel S, et al. (1996) The dielectric properties of
biological tissues: III. Parametric models for the dielectric
spectrum of tissues Phys. Med. Biol. 412271-93.

| | Frequency (MHz) | | |
|---|---|---|---|
| Tissue/fluid | 25 | 100 | 300 |
| Brain (Grey Matter) | 0.40 | 1.00 | 1.00 |
| Human Serum | 1.03 | 1.14 | 1.19 |
| Blood | 1.09 | 1.27 | 1.30 |

VEPS measurements reflect the electromagnetic properties of a volumetric composite of various tissues. The VEPS measurement will obviously depend on the properties of each component and their relative volume in the composite. Table 3 shows that at a frequency of 25 MHz the electrical conductivity of brain tissue is about 40% of that of either human serum or blood. Obviously if in part of the volume of analysis, brain tissue is replaced by serum or blood the composite volumetric impedance in the 25 MHz frequency range will be different from that of pure brain tissue. Therefore at frequencies in the range around 25 MHz the VEPS of healthy individuals should be different from that of patients with either edema (increased human serum in the analyzed volume) or hematoma (increased human blood in the analyzed volume). This is indeed what the data in Table 2 and FIG. 5 show.

FIG. 5 brings up another observation of interest. The figure shows that the β values of healthy individuals decrease with age in a correlation with a high R2 value. It is interesting to notice in FIG. 5 that at the age of 77, the β values of healthy individuals approach that of patients with a brain condition. This suggests that VEPS measurements made in the β range of frequencies alone may fail in diagnostic of brain conditions in elderly patients. It also indicates that the VEPS measurements provide insights into the more general medical condition of the human brain beyond specific disease conditions, for example as outlined herein.

Table 3 shows that at frequencies of 100 MHz to 300 MHz, the electrical properties of brain tissue are substantially more comparable to those of serum and blood than at 25 MHz and different from those at 25 MHz (the dispersion phenomena). This suggests that at frequencies of 100 MHz to 300 MHz the VEPS of healthy volunteers should be similar to that of the patients with medical conditions that affect the fluid volume in the brain. This is consistent with the results plotted in FIG. 6, which show the γ values as a function of age. FIG. 6 shows that while there is substantial statistical difference between the VEPS of patients and those of healthy volunteers in the β range of frequencies (FIG. 5, Table 2) there is no substantial statistical difference in the γ range of frequencies (FIG. 6, Table 2).

Table 3 also shows that the relative difference in electrical properties between serum and blood is larger at 300 MHz and 100 MHz, than at 25 MHz. This suggests that at these higher frequencies the VEPS should be able to discriminate between patients with edema and those with hematoma. Indeed, as shown in Table 2, despite the relative small sample size, there is a statistical difference between the VEPS of edema and hematoma patients in the frequency range of from 153 MHz to 166 MHz. This is confirmed in FIG. 6. It is evident that in the β range of frequencies there is no statistical difference between patients with edema and hematoma. On the other hand, FIG. 6 shows that in the γ range of frequencies the VEPS difference between patients with edema and hematoma is evident. FIG. 6 also shows that the correlation between the γ value and age has a different sign slope from that of the correlation between the β value and age in FIG. 5. This is an important consideration in relation to FIG. 7.

FIGS. 5 and 6 and Table 2 show that the medical condition of the brain is a function of at least two VEPS parameters, in the frequency ranges from 26 MHz to 39 MHz and the frequency ranges from 153 MHz to 166 MHz. This suggested to us that a display of data points for each subject as a function of β and γ values of the subject might provide insight into the subject brain condition. This is a typical approach in designing classifiers (Laufer, S. and Rubinsky, B. (2009) “Tissue characterization with a multimodality classifier: electrical spectroscopy and medical imaging”, IEEE Trans Biomed Eng. February; 56(2):525-8, 2009; Laufer, S, Rubinsky B (2009) Cellular Phone Enabled Non-Invasive Tissue Classifier. PLoS ONE 4(4): e5178) and FIG. 7 is a typical two-parameter scalar classifier display. The display in FIG. 7 clearly distinguishes between the different conditions of the brain. It shows that the data points for healthy individuals, those with edema and those with hematoma are found in separate β and γ value domains. The display in FIG. 7 is particularly important in relation to FIG. 5. FIG. 5 shows that the β value of healthy individuals decreases with age and approaches that of brain damaged individuals at age 77. This suggests that the detection of brain damage in the range of frequencies typical to β parameters may be less effective in older subjects than in younger subjects. However, FIG. 7 shows that the β and γ value domains inhabited by healthy, edema and hematoma patients are distinct and there are no asymptotic changes with age as in FIGS. 5 and 6. This may be serendipitous and a consequence of the fact that the correlation curves in the β and γ curves with age have different sign slopes. Therefore the effect of age is cancelled in a display in terms of β and γ values and only the effect of the medical condition remains. FIG. 7 illustrates the promise in building VEPS multifrequency classifiers for non-contact diagnosis of diseases.

It is known from clinical studies that the changes in the diseased brain are complex and occur over periods of time. From the data we anticipate that the VEPS in a patient with a medical condition in the brain will vary in time following the pattern observed here. Therefore measuring VEPS of a patient suspected to have a medical condition in the brain may also be used to determine if a patient should be sent to medical imaging at a central facility.

In summary, this clinical study on VEPS multifrequency measurements in patients with edema and hematoma in the brain and in healthy volunteers demonstrates that VEPS of patients with medical conditions of edema and hematoma in the brain is statistically different from that of healthy volunteers, and that it will be possible to use a simple device and a classifier display for the diagnosis of medical conditions in tissues, e.g., the brain. The ability to distinguish edema in the brain from hematoma is an important finding. First, it points to the sensitivity of VEPS. More important, the ability to differentiate between edema and hematoma at an early stage and even before the patient is brought to the medical imaging facility at the central hospital is of great clinical importance, as it may affect the acute treatment modality.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of obtaining a VEPS tissue signature, comprising:
positioning a tissue between a first induction coil and a second induction coil;
driving an alternating current in a frequency range through the first induction coil;
measuring the alternating current produced in the second induction coil at the frequency range; and
determining a phase shift of the alternating current between the first induction coil and the second induction coil at the frequency range to obtain a VEPS tissue signature.

2. The method according to claim 1, wherein the frequency range is within between 1 Hz and 1 THz.

3. The method according to claim 2, wherein the frequency range is in the range of between 1 KHz to 20 GHz.

4. The method according to claim 3, wherein the frequency range is within between 0.1 MHz and 150 MHz.

5. The method according to claim 2, wherein the frequency range is in the range of between 1 KHz to 20 GHz.

6. The method according to claim 5, wherein the frequency range is within between 100 MHz and 500 MHz.

7. The method according to claim 1, further comprising:
driving an alternating current in a second frequency range through the first induction coil,
measuring the alternating current produced in the second induction coil at the second frequency range,
determining a phase shift of the alternating current between the first induction coil and the second induction coil at the second frequency range, and
obtaining a VEPS tissue signature based on the first frequency range and the second frequency range.

8. The method according to claim 7, wherein the second frequency range is in the range of between 1 Hz and 1 THz.

9. The method according to claim 8, wherein the second frequency range is in the range of between 1 KHz to 20 GHz.

10. The method according to claim 9, wherein the second frequency range is in the range of between 0.1 MHz and 150 MHz.

11. The method according to claim 8, wherein the second frequency range is in the range of between 1 KHz to 20 GHz.

12. The method according to claim 11, wherein the second frequency range is in the range of between 100 MHz and 500 MHz.

13. The method according to claim 1, wherein the first and second induction coils do not contact the tissue.

14. The method according to claim 1, wherein the tissue is selected from the group consisting of: brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, prostate tissue, breast tissue, esophagus tissue, GI tract tissue and trunk tissue.

15. A method for providing a determination of the condition of a tissue in a subject, the method comprising:
obtaining a VEPS tissue signature, and
determining the condition of a tissue in a subject based on the tissue signature.

16. The method according to claim 15, wherein the condition is selected from the group consisting of: edema, hemorrhage, hematoma, ischemia, dehydration, the presence of a tumor, infection, brain degeneration, extravasation, internal bleeding, maternal hemorrhage, and tissue health relative to age.

17. The method according to claim 15, wherein the determining step comprises:
comparing the VEPS tissue signature to a reference, and
providing a determination based on the comparison.

18. The method according to claim 17, wherein the comparing comprises graphically plotting the tissue signature relative to a panel of classifiers.

19. The method according to claim 15, further comprising determining a clinical parameter.

20. The method according to claim 19, wherein the clinical parameter is the age of the subject.

21. The method according to claim 15, wherein the determination is used to provide a diagnosis for the subject, wherein the method further comprises:
providing a diagnosis for the subject based on the determination of the condition of the tissue.

22. The method according to claim 15, wherein the determination is used to provide a prognosis for the subject, wherein the method further comprises:
providing a prognosis for the subject based on the determination of the condition of the tissue.

23. The method according to claim 15, wherein the determination is used to monitor a subject's health or responsiveness to a therapeutic treatment, wherein the method further comprises:
obtaining a second VEPS signature at a second point in time, and
monitoring the subject's health or responsiveness to a therapeutic treatment based on the determination of the first VEPS signature and the second VEPS signature.

24. A system for obtaining a VEPS tissue signature, comprising:
a first induction coil and a second induction coil positioned opposite one another; and
a measurement system operably connected to the second induction coil, wherein the measurement system is configured to measure a phase shift of one or more alternating currents between the first and second induction coil at one or more frequencies in two or more frequency ranges.

25. The method according to claim 24, wherein at least one of the two or more frequency ranges is in the range of between 1 Hz and 1 THz.

26. The method according to claim 25, wherein at least one of the two or more frequency ranges is in the range of between 1 KHz to 20 GHz.

27. The method according to claim 26, wherein at least one of the two or more frequency ranges is in the range of between 0.1 MHz and 150 MHz.

28. The method according to claim 25, wherein at least one of the two or more frequency ranges is in the range of between 1 KHz to 20 GHz.

29. The method according to claim 28, wherein at least one of the two or more frequency ranges is in the range of between 100 MHz and 500 MHz.

30. The method according to claim 24, wherein the first and second induction coils do not contact the tissue.

31. The method according to claim 24, wherein the tissue is selected from the group consisting of: brain tissue, lung tissue, heart tissue, muscle tissue, skin tissue, kidney tissue, cornea tissue, liver tissue, abdomen tissue, head tissue, leg tissue, arm tissue, pelvis tissue, chest tissue, prostate tissue, breast tissue, esophagus tissue, GI tract tissue and trunk tissue.

32. The system according to claim 24, further comprising a data processor module configured to calculate VEPS values from the plurality of frequencies corresponding to the two or more frequency ranges.
